(19) United States
(12) Patent Application Publication
(10) Pub. No.: US 2010/0174187 A1
(43) Pub. Date: Jul. 8, 2010

(54) METHOD AND APPARATUS FOR DETERMINING HYDRATION LEVELS

(75) Inventors: Eric Cohen-Solal, Ossining, NY (US); Yan S. Shi, Sleepy Hollow, NY (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 12/294,661

(22) PCT Filed: Mar. 29, 2007
(86) PCT No.: PCT/IB2007/051136

§ 371 Date: Sep. 26, 2008

Related U.S. Application Data
(60) Provisional application No. 60/788,453, filed on Mar. 31, 2006.

(51) Int. Cl. A61B 8/00

(52) U.S. Cl. 600/438

(57) ABSTRACT

A method and apparatus for determining patient hydration levels is disclosed. The method includes measuring a thickness of a dermis layer.

Human beings and animals rely on water to live. Water is essential to many biological and biochemical reactions that take place. As a result it is important maintain a minimum amount of water in the body.

Water is exchanged dynamically between the body and the environment. Under normal conditions, body fluids are well maintained in terms of both electrolyte concentrations and volume through processes like drinking, urine production and sweating. However, fluid balance may be disturbed due to a variety of reasons, including, but not limited to: insufficient water intake due to conditions such as chronic hypodipsia; gastrointestinal losses due to illness; renal conditions; skin losses; clinical procedures such as hemodialysis.

All of these conditions can result in excessive fluid loss, and attendant dehydration or fluid deficit. More generally, dehydration refers to water loss with or without accompanied electrolyte loss, particularly sodium. Fluid loss of only a few percent of body weight causes discomfort and impaired body function. As dehydration levels increase, patients become fatigued and irritable, with symptoms of dry mouth, less-frequent urination and tachycardia.

Without proper water replenishment, fluid deficit can eventually develop to a clinical emergency when fluid loss is greater than 9% of body weight, resulting in organ damages, coma, even death.

From the above, it can be appreciated that the early identification of dehydration followed by prompt and adequate fluid intake can substantially reduce the risk of severe dehydration, and the potentially severe complications thereof.

Commonly, the clinical assessment of level of hydration is mainly based on physical examination. Symptoms of dehydration include dry mouth and mucous membrane, sunken eyes, orthostatic hypotension, delayed capillary refill, and poor skin turgor. However, the clinical assessments can be subjective and have a low sensitivity and specificity in general.

Laboratory tests on blood and urine samples have also been used to determine hydration levels. Typically, the tests are performed after physical assessment of dehydration symptoms in order to generate additional information; validate the diagnosis; and to aid in the treatment of the patient. An advantage of these tests over physical examination is that they provide objective and quantitative measurements. Nevertheless, the laboratory tests require special lab equipment and are usually time-consuming and costly.

There is a need, therefore, for a method and apparatus adapted to provide an accurate measure of hydration levels in patients that overcome at least some of the shortcomings described above.

In accordance with an example embodiment, an apparatus includes a transducer operative to transmit mechanical waves and to receive reflections of the mechanical waves. The apparatus also includes a receiver operative to receive electrical signals corresponding to the reflections. In addition, the apparatus includes a processor operative to calculate a distance corresponding to each of the reflections, wherein the distance is representative of a thickness of a dermis layer.

In accordance with an example embodiment, a method includes transmitting mechanical waves from an epidermis into a body; receiving a reflection of the mechanical waves; calculating a distance from a contact location with the body to a reflection location; determining a thickness of a dermis layer from the distance; and based on the thickness, determining a hydration level of the body.

In accordance with another example embodiment, an apparatus includes a dehydration sensor operative to contact one surface of a body and including a plurality of transducers, each of which is operative to transmit mechanical waves and to receive reflections of the mechanical waves. The apparatus also includes a receiver operative to receive electrical signals corresponding to the reflections; and a processor operative to calculate a distance corresponding to each of the reflections, wherein the distance between a stratum corneum layer and a subcutaneous fat (SF) layer.

As used herein, the terms ‘a’ and ‘an’ mean one or more; and the term ‘plurality’ means two or more.

As used herein, the term ‘patient’ includes humans, mammals and fish.

The present teachings are best understood from the following detailed description when read with the accompanying drawing figures. It is emphasized that the various features are not necessarily drawn to scale. In fact, the dimensions may be arbitrarily increased or decreased for clarity of discussion.

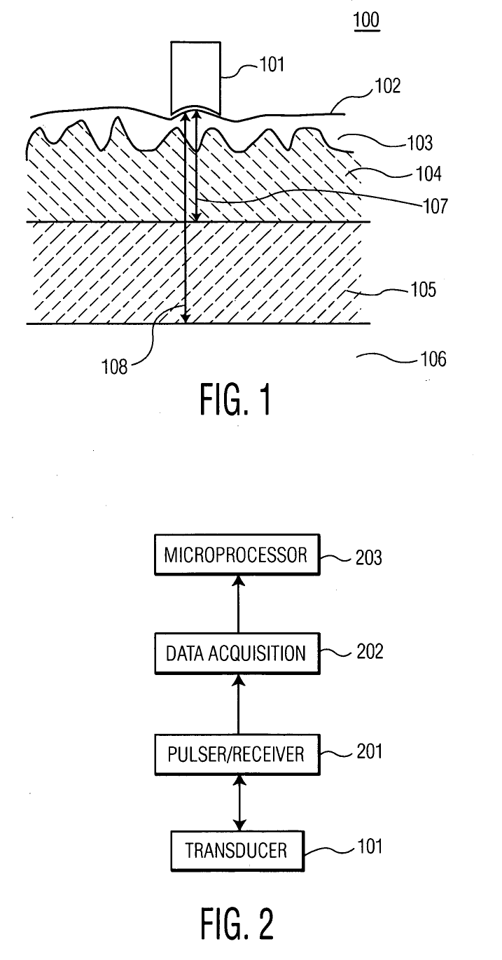

FIG. 1 is a cross-sectional view a transducer contacting the surface of a body in accordance with an example embodiment.

FIG. 2 is a simplified block diagram an apparatus in accordance with an example embodiment.

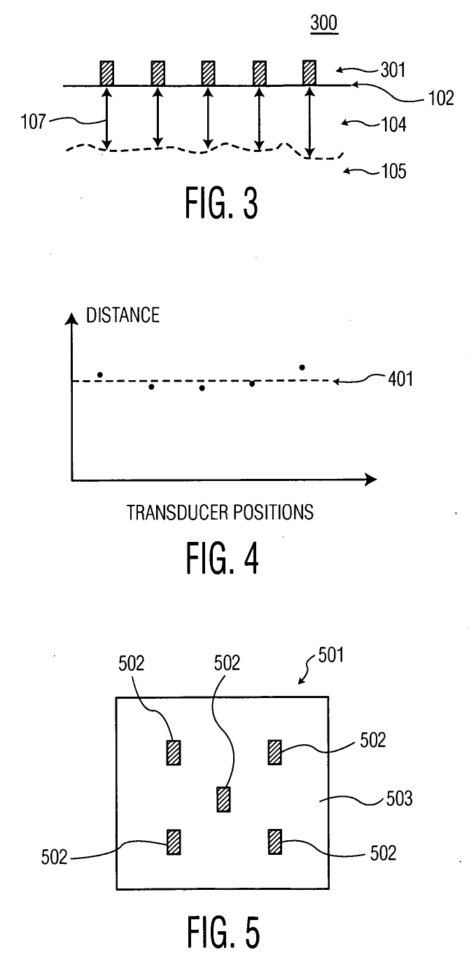

FIG. 3 is a cross-sectional view of an apparatus in accordance with another example embodiment.

FIG. 4 is a graphical representation of distance measurements in accordance with an example embodiment.

FIG. 5 is a top-view of a dehydration sensor in accordance with another example embodiment.

In the following detailed description, for purposes of explanation and not limitation, example embodiments disclosing specific details are set forth in order to provide a thorough understanding of the present teachings. However, it will be apparent to one having ordinary skill in the art having had the benefit of the present disclosure that other embodiments that depart from the specific details disclosed herein. Moreover, descriptions of well-known devices, hardware, software, firmware, methods and systems may be omitted so as to avoid obscuring the description of the example embodiments. Nonetheless, such hardware, software, firmware, devices, methods and systems that are within the purview of one of ordinary skill in the art may be used in accordance with the example embodiments. Finally, wherever practical, like reference numerals refer to like features.

The example embodiments described relate primarily to A-scans. A-scan refers to a measurement technique where a transducer transmits mechanical waves into an object and the amplitudes of the reflected mechanical waves are recorded as a function of time. Normally, the transducer is located on one surface of the object and only structures that lie along the direction of propagation of the mechanical waves are interrogated. As reflected waves (echoes) return from interfaces within the object or tissue, the transducer converts the mechanical wave into a voltage that is proportional to the echo intensity.

FIG. 1 is a cross-sectional view of an apparatus 100 in accordance with an example embodiment. The apparatus 100 includes a transducer 101 and electronics (not shown in FIG. 1). The transducer 101 is illustratively an ultrasonic transducer that emits mechanical waves having a frequency illustratively in the range of approximately 1.0 MHz to approximately 20.0 MHz. In a specific embodiment, the transducer is unfocused. Alternatively, the transducer 101 is a focused transducer.

In an illustrative embodiment, the transducer 101 is a single element transducer that operates in a pulsed mode. Accordingly, a relatively wide bandwidth, greater than approximately 25% is desirable. In addition, the frequency of the transducer is usefully relatively high to foster measurement accuracy. In a specific embodiment, the transducer 101 is adapted to operates in the range of approximately 5.0 MHz to approximately 20.0 MHz. In an alternative embodiment, a relatively low frequency transducer can be implemented as transducer 101.

The transducer 101 may be one of a variety of types of transducers useful in medical applications and known to those skilled in the art. For example, the transducer 101 may be a piezoelectric element such as a lead zirconate titanate (PZT) element. By way of another example, the transducer may be a piezoelectric micromachined ultrasonic transducer (PMUT). It is emphasized that the PZT and PMUT transducers are merely illustrative and that other transducers are contemplated.

The transducer 101 is in contact with a stratum corneum layer 102 of the patient. A known coupling material (e.g., coupling gel) may be applied to the location of layer 102 where contact is made. This layer 102 comprises a layer of ‘dead’ skin having a thickness of approximately 0.010 mm to approximately 0.020 mm. Optionally, the transducer protrudes into a layer of living epidermis 103 of the body of a patient, although does not puncture the layer 102. The living layer of epidermis 103 is relatively thin, or the order of approximately 0.030 mm to approximately 0.130 mm.

The patient may be a human being, and the apparatus is a medical testing device. It is contemplated that the apparatus be used in veterinary testing of animals where concerns about dehydration require a measure of hydration levels.

Beneath the epidermis layer 103 is a dermis layer 104. Illustratively, the dermis layer has a thickness of approximately 1.1 mm. As described more fully herein, the apparati and methods of the illustrative embodiments usefully measure the thickness of the dermis layer 104 and from these measurements garner a relative measure of hydration for each patient.

Beneath the dermis layer 104 is a layer of subcutaneous fat (SF) 105. Illustratively, this layer is on the order of approximately 1.2 mm in thickness. As described herein, in an example embodiment reflections of mechanical waves (e.g., ultrasound waves) at the interface of the dermis layer 104 and the SF layer 105 are used to determine the thickness of the dermis layer 104.

Finally, beneath the SF layer 105 is a layer 106 of muscle, or bone, or both.

It is emphasized that the layer thicknesses provided above are merely illustrative. To this end, the layer thicknesses may vary widely from location to location on the body and from patient to patient. In addition, a patient's posture may impact the thicknesses of some of the layers. Furthermore, medical conditions can impact the thicknesses of the layers. Notably, the dermis layer acts as a fluid (e.g., water) reservoir for the human body. As the body becomes dehydrated, the amount of fluid in the dermis is reduced. As such, the dermis thickness is reduced and provides an indication of the level of dehydration. Example embodiments exploit this characteristic of the dermis layer to determine the level of dehydration by measuring the thickness of this layer and comparing this to a baseline value at acceptable hydration levels.

Mechanical waves are emitted from the transducer 101 and propagate through the tissue layers 102-104. In the present example embodiment, the mechanical waves are reflected from at the surface of the SF layer 105 and traverse the tissue. The reflected waves (or echoes) are incident on the transducer 101. Alternatively, the mechanical waves are reflected from the layer 106. In either case, the mechanical waves are then converted into electrical signals by the transducer 101 and processed as described more fully herein.

In a specific embodiment, the apparatus 100 is adapted to calculate a distance 107, which represents a thickness of the dermis layer 104, neglecting the very thin layers 102, 103. In an alternative embodiment, the apparatus 100 is adapted to measure a thickness 108, which represents the thickness of the dermis layer 104 and the SF layer 105.

The distances 107 and 108 are calculated simply by multiplying the velocity of the mechanical waves in the layers 102-104 and layers 102-105, respectively, by the time between the transmission of a mechanical wave (or pulse) from the transducer and the reception of the reflected mechanical wave. Notably, the speed of mechanical waves in the layers 102-105 is substantially constant, being substantially independent of the frequency of the mechanical wave, the temperature of the skin and tissue, and the hydration level of the skin and tissue. Accordingly, the distances 107, 108 can be calculated using the estimated constant velocity.

It is contemplated that the temperature, the frequency of the mechanical waves and the hydration levels be accounted for in the determination of the velocity. For example, from a baseline velocity in skin and tissue at a particular temperature and baseline hydration level of the skin and tissue, the velocity of the mechanical waves may be adjusted by approximately 0.10%/° C. and approximately 0.20% per 1.0% water loss with respect to body weight. After these adjustments are made the simple calculation may be made to determine the distances 107 and thus the thickness of the dermis layer 104; or the distance 108 and thus the thickness of the dermis layer 104 and the SF layer 105.

In the interest of simplicity, the description of the function of the apparatus 101 concentrates on the measurement of the distance 107. Of course the principles described can be readily applied to the measurement of the distance 108.

In accordance with an example embodiment, a plurality of measurements is made of the distance 107. In another example embodiment, measurements are made in a plurality of locations. The measurements are beneficially made at a location of the body where there is little, if any, deformable tissue. To this end, in order to ensure proper coupling between the transducer 101 and the skin 102, pressure must be applied. However, pressure can alter the thickness of the measure if the material being measured is too deformable to allow for accurate and consistent measurements. As such, locations such as the forehead, the sternum and the tibia on humans are useful in exacting an accurate measurement of the distance 107.

In addition, errors in thickness measurements may be significantly avoided by including a pressure sensor at the transducer to ensure that the same pressure is consistently applied to the body in each measurement, or by allowing on the weight of the transducer to be applied at the location of the body, or both.

As described more fully herein, the measured distance 107 is compared to a baseline distance for a particular patient at the same location. If the distance 107 is less than the baseline distance, a level of dehydration has occurred. As can be appreciated, by comparing the measured thickness of the dermis to the baseline distance, the measurements can be used to assess excessive fluid conditions in a patient

FIG. 2 is a simplified schematic block diagram of an apparatus 200 in accordance with an example embodiment. Many features of the apparatus 200 are common to those of the apparatus described in connection with the example embodiments of FIG. 1. These common details may not be repeated in order to avoid obscuring the description of the present example embodiments.

The transducer 101 is connected to a pulse generator and receiver (PGR) 201. The PGR 201 transmits electrical pulses of finite duration and at a chosen periodicity to the transducer 101. The electrical pulses are converted to mechanical pulses that are emitted by the transducer 101 into layers 102-104.

The reflected mechanical waves are converted into electrical signals by the transducer 101 and transmitted to the PGR 201. The PGR 201 includes a receiver circuit, filters and amplifiers. The amplifiers are useful particularly when the mechanical waves are relatively high frequency, as the attenuation of the mechanical wave in the layers 102-105 increases with increasing frequency. Suitable receiver circuits, filter circuits and amplifier circuits are well known to those skilled in analog signal processing.

The apparatus 200 also includes a data acquisition module 202. The module 202 illustratively includes a register or memory adapted to store received signal data from received from the PGR 202. For example, the module 202 may be an engine that calculates the time of flight of the transmitted mechanical wave for each transmitted pulse.

These data are then stored for calculating the distance 107. A processor/microprocessor 203 is provided to effect various functions of the apparatus 200. The microprocessor 203 may be a commercially available microprocessor such as a Pentium® from Intel Corporation, or another suitable processor. The processor 203 optionally includes operating system (OS) software. The processor 203 includes application code written to effect the algorithms described herein. Such code is within the purview of one of ordinary skill in the art.

In an example embodiment, the processor 203 is adapted to implement data capture and carry out correlation algorithm. Illustratively, the data capture includes analog to digital (A/D) conversion of received electrical signals, and storage of the data. Illustratively, the time of flight is determined based on a correlation algorithm.

Alternatively, the time of flight may be determined via edge detection, such as positive/negative slope, zero-crossing techniques known in the art. Notably, it is beneficial to perform a calibration sequence against a known distance using the selected algorithm. This ensures greater accuracy and consistency of the measurements.

It is emphasized that the noted algorithms are known to those skilled in the art and that other algorithms for determining the time-of-flight and distance/thickness measurements are contemplated.

In operation, upon receiving an input from an operator, the processor 203 triggers the transmission of pulses by the PGR 202 to begin a test. The processor 203 algorithmically determines the distance 107 by retrieving the time of flight of a pulse and multiplying the time by the velocity. The processor 203 may also determine an average distance from a plurality of measurements at the same location; or a total distance from a plurality of measurements from different locations; or an average total distance from a plurality of measurements from each of a plurality of locations.

Illustratively, many measurements from transmitted and reflected pulses may be effected at the same location and from these an average of the distance 107 may be determined. In a specific embodiment, more than five measurements are used to calculate an average.

Alternatively, pulses may be transmitted at more than one location (e.g., the forehead, the sternum and the tibia) and a total thickness determined. Multiple measurements can be made and an average of the total calculated. In a specific embodiment, more than five measurements at each location are used to calculate an average for each of the locations. The average values and average of the average values (referred to as a total average) may also be stored in the data acquisition module 202.

As can be appreciated, the posture of a patient can impact the measurement. To this end, the thickness of the skin at a location on the body can be impacted by the posture. For example, a thickness of skin at a person's mid-section can be compacted when sitting resulting in a different measurement than when standing. Beneficially, by garnering a total thickness from the multiple locations of the body, some degree of compensation is provided for posture-induced skin thickness variation.

In an example embodiment, the processor 203 may receive the temperature of the skin 102 and tissue, or the frequency of the transmitted mechanical wave, or the hydration level from a recent measurement, or a combination of these variables to adjust the velocity to account for these variables.

After calculating the distance 107 or an average distance as described above, the processor 203 algorithmically compares the distance 107 or the average distance of the most recent measurement with the baseline. From these comparisons, a relative measure of hydration levels can be made. Alternatively, the processor may algorithmically determine the fluid hydration level of a patient by comparing the measurement data to the baseline levels.

In accordance with an example embodiment, multiple measurements are performed in order to compile a database useful in determining the relationship between skin thickness changes and dehydration levels at specific location for each patient. These measurements provide a baseline hydration value at acceptable (e.g., healthy) hydration levels that can then be used to map the measurements to the fluid hydration level in the patient. Alternatively or additionally, the baseline may be determined from data from a large group of patients. These population-based baselines can be further compiled demographically so that a particular patient's hydration levels can be compared to acceptable fluid levels of people of similar height, weight, age and other similar criteria.

FIG. 3 is a cross-sectional view of an apparatus 300 in accordance with an example embodiment. Many features of the apparatus 300 are common to those of the apparati described in connection with the example embodiments of FIGS. 1 and 2. These common details may not be repeated in order to avoid obscuring the description of the present example embodiments.

The apparatus includes a plurality of transducers 301. The transducers 301 are substantially identical to transducer 101 and may be coupled to the PRG 201 via a multiplexer (not shown) or through a PRG 201 having multiple inputs. The data from the transducers are individually processed as described previously.

Each transducer 301 rests on one surface of a patient and transmits mechanical waves into the body. These waves traverse the layers 102, 103, and are reflected from the SF layer 104. As noted previously, the dermis 103 provides a relatively accurate measure of the hydration level of the body. Accordingly, the apparatus provides a measure of the thickness of the dermis and thus the hydration levels of a patient.

In the present embodiment, each transducer 301 measures a respective thickness 107 of the dermis. The measurements include the transmission, reflection and reception of mechanical waves as described previously. From measured time of flight the distance 107 and thus the thickness of the dermis 104 may be determined at each transducer location by the processor 203. Notably, the thickness 108 may also be measured by similar technique.

Optionally, a plurality of measurements may be made and an average fit compiled at the processor. In addition, the plurality of transducers 301 may be used at multiple locations and, optionally, a plurality of measurements may be made at each location.

The use of multiple transducers 301 provides greater levels of accuracy as multiple measurements are made in a localized area. This accuracy can be improved further by making multiple measurements. In addition to other benefits, local variations in the thickness of the dermis 302 may be accounted for through a relatively straight-forward averaging process described presently.

FIG. 4 is a graphical representation of the distance 107 versus position of the respective transducers 301. A line 401 represents a mathematical ‘fit’ (e.g., least squares-fit) of the distance data over the area of the body being measured. This fit provides a mean value and standard deviation from the mean for the area of the body being tested by the multiple transducers. Notably, a plurality of measurements may be made and a composite fit determined. In addition, multiple measurements may be made at multiple locations and composite fit can be determined for each location. These measurement data can be stored in the data acquisition module 202.

A baseline value of each patient at suitable hydration may be calculated using the transducers 304 in a similar manner. Beneficially, the baseline is determined when subjects are well hydrated through drinking water or other fluids, or by intravenous fluid injection. The quantity of fluid may be recommended by nutritionists or other medical practioners under normal circumstances. In a specific embodiment, the baseline measurements are carried out over a preset number of days to ensure its reliability. The average of all measurements may then be used as a baseline.

The measurement data stored in the module 202 can be compared to the baseline value for a determination of the level of dehydration in the patient. In an example embodiment, the measurement data from multiple locations may be used to determine the dehydration level. Moreover, averages of the various distance measurements described previously may be used to determine the dehydration level.

Using the transducers 301, a plurality of thickness measurements may be made and provided graphically as in FIG. 4. These respective averages or fits of the distance data from each measurement may be averaged to provide a composite average. This composite average reduces inaccuracies in measurement by taking multiple measurements at the same locations over a particular area of the body. Beneficially, local variations are suitably accounted for.

FIG. 5 is a top view of a dehydration sensor 501 in accordance with an example embodiment. The sensor 501 includes many features common to those of the apparati described in connection with the example embodiments of FIGS. 1-4. These common details may not be repeated in order to avoid obscuring the description of the present example embodiments.

The sensor 501 includes a plurality of transducers 502 arranged in a substrate 503. In an example embodiment, the transducers 502 are substantially the same as the transducers 101, 303 described previously. The substrate 503 may be one of a variety of known materials that prevents mechanical and electrical interference between the transducers 502. Page: 15 Beneficially, the substrate 503 is a layer that assures uniform contact between the transducers 502 and the skin. The substrate 503 should be substantially acoustically transparent (i.e., have a similar acoustic impedance as the skin) and relatively thin.

The sensor 501 is adapted to provide a plurality of measurements over a region of the body in a manner similar to that described in connection with the example embodiments of FIGS. 3 and 4. The transducers 502 of the sensor 501 may be coupled to the PRG 201 via a multiplexer (not shown) or through a PRG 201 having multiple inputs. The data from the transducers are individually processed as described previously.

Each transducer 502 rests on one surface of a patient and transmits mechanical waves into the body. These waves traverse the epidermis 102 and dermis 104, and are reflected from a lower tissue layer, the SF layer 105. As noted previously, the dermis 104 provides a relatively accurate measure of the hydration level of the body. Accordingly, the apparatus provides a measure of the thickness of the dermis and thus the hydration levels of a patient.

In a specific embodiment, each transducer 502 measures a respective thickness 104 of the dermis. The measurements include the transmission, reflection and reception of mechanical waves as described previously. From measured time of flight the distance 107 and thus the thickness of the dermis 104 may be determined at each transducer location by the processor 203.

The sensor 501 may also garner measurements from a number of locations on the body as described. Moreover, a plurality of measurements may be made at each location and an average for each calculated to garner the total average as described previously.

The measurement data stored in the module 202 can be compared to the baseline value for a determination of the level of dehydration in the patient. In an example embodiment, the measurement data from multiple locations may be used to determine the dehydration level. Moreover, average distance measurements described previously may be used to determine the dehydration level.

In view of this disclosure it is noted that the various methods and devices described herein can be implemented in hardware and software. Further, the various methods and parameters are included by way of example only and not in any limiting sense. In view of this disclosure, those skilled in the art can implement the present teachings in determining their own techniques and needed equipment to effect these techniques, while remaining within the scope of the appended claims.

1. An apparatus (100, 200, 300), comprising:
a transducer (101,301) operative to transmit mechanical waves and to receive reflections of the mechanical waves;
a receiver (201) operative to receive electrical signals corresponding to the reflections;
a processor (203) operative to calculate a distance (107,108) corresponding to each of the reflections, wherein the distance is representative of a thickness of a dermis layer.

2. An apparatus as recited in claim 1, wherein the distance is a distance to a boundary of a dermis layer (104) and a subcuneous fat (SF) layer (105) and the distance is proportional to a dehydration level in a body.

3. An apparatus as recited in claim 2, wherein the processor is operative to garner a plurality of calculations from the transducer and to calculate an average distance.

4. An apparatus as recited in claim 3, wherein the processor is operative to compare the average distance with a baseline distance for a patient and to calculate the dehydration level based on the average distance.

5. An apparatus as recited in claim 1, wherein the mechanical waves are ultrasonic waves.

6. An apparatus as recited in claim 3, wherein the plurality of calculations from the transducer are from more than one location on a body.

7. A method, comprising:
transmitting mechanical waves into body;
receiving a reflection of the mechanical waves from a subcutaneous fat (SF) layer (105);
calculating a distance (107) from a contact location on the body to a reflection location;
determining a thickness of a dermis layer (104) from the distance; and
based on the thickness, determining a hydration level of the body.

8. A method as recited in claim 7, wherein the distance is a distance between a stratum corneum layer (102) and the SF layer.

9. A method as recited in claim 7, further comprising providing a plurality of transducers (301) at an area where the mechanical waves are introduced into the body, and each of the transducers is operative to emit the mechanical waves and to receive the reflections.

10. A method as recited in claim 7, further comprising performing the transmitting and the receiving at least twice; performing the determining for each of the distances; and calculating an average thickness of the dermis layer.

11. A method as recited in claim 10, wherein the determining the hydration level further comprises comparing the average thickness with a baseline thickness for a patient.

12. A method as recited in claim 7, further comprising performing the transmitting and receiving at a plurality of locations on the body.

13. A method as recited in claim 12, further comprising:
performing the determining for each of the plurality of locations; and
calculating an average thickness for each location; and
calculating a total average thickness from the average thickness from each location, wherein the determining the hydration level further comprises comparing the total average thickness with a baseline thickness.

14. An apparatus (200), comprising:
a dehydration sensor (501) operative to contact one surface of a body and including a plurality of transducers (502), each of which is operative to transmit mechanical waves and to receive reflections of the mechanical waves;
a receiver (201) operative to receive electrical signals corresponding to the reflections; and
a processor (203) operative to calculate a distance (107) corresponding to each of the reflections, wherein the distance is a distance between a stratum corneum layer (102) and a subcutaneous fat (SF) layer (105).

15. An apparatus as recited in claim 14, wherein the distance is representative of a thickness of a dermis layer (104) and the distance is proportional to a dehydration level in a body.

16. An apparatus as recited in claim 15, wherein the processor is operative to garner a plurality of the calculations and to calculate an average distance.

17. An apparatus as recited in claim 17, wherein the processor is operative to compare the average distance with a baseline distance for a patient and to calculate the dehydration level based on the average distance.

18. An apparatus as recited in claim 14, wherein the mechanical waves are ultrasonic waves.

19. An apparatus as recited in claim 16, wherein the plurality of calculations are from more than one location on a body.

20. An apparatus as recited in claim 14, wherein the each of the plurality of transducers is a single transducer.
